(19) United States
(12) Patent Application Publication
(10) Pub. No.: US 2025/0114447 A1
(43) Pub. Date: Apr. 10, 2025

(54) VACCINE FOR EQUINE HERPESVIRUS

(71) Applicant: Intervet Inc., Rahway, NJ (US)

(72) Inventors: Sylvia Reemers, Den Bosch (NL); Saskia Van de Zande, Geel (BE)

(73) Assignee: Intervet Inc., Rahway, NJ (US)

(21) Appl. No.: 18/294,583

(22) PCT Filed: Aug. 5, 2022
(86) PCT No.: PCT/EP2022/072079

§ 371 Date: Feb. 2, 2024

(30) Foreign Application Priority Data
Aug. 6, 2021 (EP) 21190028.7

(51) Int. Cl. A61K 39/245; A61K 39/00; A61P 37/04

(52) U.S. Cl. CPC A61K 39/245; A61P 37/04; A61K 2039/54; A61K 2039/543; A61K 2039/545; A61K 2039/552; A61K 2039/55577

(57) ABSTRACT

The present invention is directed to an immunogenic composition comprising an antigen of Equine Herpes Virus type 1 (EHV-1) and an antigen of Equine Herpes Virus type 4 (EHV-4). The immunogenic composition reduces viremia and other clinical reactions in horses infected with EHV-1 when compared to non-treated horses but also when compared to EHV-1 vaccinated horses.

The present invention relates to the field of animal health and in particular of Equine Herpes Viruses (EHV). The invention also relates to immunogenic compositions comprising the combination of EHV-1 and EHV-4 viruses.

### BACKGROUND

Major economic losses to the equine industry result from infection by two species of equine herpesvirus. These two equine herpesvirus species, EHV-1 and EHV-4, belong to the herpesvirus sub-family alpha-herpesvirus. EHV-1 is the major cause of virus-induced abortion in equines and causes respiratory and neurological disease. EHV-4 can also induce respiratory symptoms, abortions or neurological disorder. Both viruses enter via the respiratory tract where a first lytic replication takes place in the respiratory epithelium. After primary replication in the nasal mucosa, EHV-1 but not EHV-4 is capable of efficient infection of leukocytes present in the tributary lymphoid tissue, which then results in a leukocyte-associated viremia. From there, EHV-1 is spread and can reach the end vessels of the pregnant uterus or the central nervous system (CNS).

It is thus important to minimize viremia. It is also important to provide a safe and efficacious vaccine against an infection of EHV-1, against an infection of EHV-4 and/or against and infection of EHV-1 and EHV-4. Furthermore, preferably the vaccine provides increased protection against infection of EHV-1, and/or EHV-4. Also preferably the vaccine reduces the clinical signs of the infection. In addition, it is advantageous to provide a vaccine that needs less antigen but is effective against infection against EHV-1, or EHV 4 or against EHV-1 and EHV-4.

Surprisingly it was found that vaccination with a combination of EHV-1 and EHV-4 reduces viremia of EHV-1 after EHV-1 infection. Upon vaccination of horses with EHV-1 or a combination EHV-1 and EHV-4 (EHV 1_4) vaccine there is a reduction in EHV-1 viremia compared to unvaccinated controls after EHV-1 challenge infection. This reduction in viremia is higher in horses vaccinated with EHV 1_4 vaccine than in horses vaccinated with only EHV-1 vaccine. This is surprising as viremia is caused by EHV 1 and not by EHV 4. Even more surprising is that the combination vaccine EHV 1_4 had a much lower antigen concentration and the EHV-1 vaccine while still having a better effect on the reduction of the viremia. Not only the amount of EHV 1 in the EHV 1_4 was lower than the amount of EHV 1 in the EHV-1 vaccine, but the total amount of antigen, i.e. EHV 1 and EHV 4, was lower.

Heldens et al (Vaccine 19 (2001) 4307-4317) evaluated the efficacy of an inactivated EHV 1 and EHV 4 whole virus vaccine (Duvaxyn EHV_{1,4}) and found that vaccinated animals showed reduction in clinical signs and virus excretion, when compared to unvaccinated control animals. It was found that viremia in the vaccinated group was shorter and less frequent than in the un-vaccinated group. No comparison with EHV-1 vaccinated animals was made.

Also it was found that the EHV 1_4 provides a better protection against infection of EHV-1 than non-vaccinated controls and when vaccinated with a vaccine comprising EHV-1 but not EHV-4. It was found that the overall clinical reaction was reduced and that the peak of the clinical reaction was reduced. In addition, it was surprisingly found that less antigen per dose was needed to obtain the above-mentioned protection and reduction in viremia when compared to a vaccine comprising EHV-1 but not EHV-4.

### SUMMARY OF INVENTION

In a first aspect the invention is directed to an immunogenic composition comprising an antigen of Equine Herpes Virus type 1 (EHV-1) and antigen of Equine Herpes Virus type 4 (EHV-4).

In another aspect the invention is directed to an immunogenic composition comprising an antigen of Equine Herpes Virus type 1 (EHV-1) and an antigen of Equine Herpes Virus type 4 (EHV-4) for use in reducing viremia of EHV-1 in an animal. Alternatively, the invention is directed to a method to reduce viremia in an animal comprising administering an immunogenic composition comprising an antigen of Equine Herpes Virus type 1 (EHV-1) and an antigen of Equine Herpes Virus type 4 (EHV-4) to an animal.

The reduction in viremia is an improvement in comparison to animals of a non-treated control group of the same species, or to animals of the same species that are treated with an immunogenic composition comprising an antigen of EHV-1 but not an antigen of EHV-4.

The reduction of viremia may be selected from the group consisting of a shorter duration of viremia, an earlier end to viremia, a later onset of viremia, a lower virus load, or a lower peak viral load and combinations thereof. Suitably the reduction in viremia is a reduction in total viral load and/or a reduction in the peak viral load.

In another aspect the invention is directed to an immunogenic composition comprising an antigen of Equine Herpes Virus type 1 (EHV-1) and an antigen of Equine Herpes Virus type 4 (EHV-4) for use in reducing clinical reaction of EHV-1 infection in an animal. Alternatively, the invention is directed to a method to reduce clinical reaction of EHV-1 infection comprising administering an immunogenic composition comprising an antigen of Equine Herpes Virus type 1 (EHV-1) and an antigen of Equine Herpes Virus type 4 (EHV-4) to an animal.

In another aspect the invention is directed to an immunogenic composition comprising an antigen of Equine Herpes Virus type 1 (EHV-1) and an antigen of Equine Herpes Virus type 4 (EHV-4) for use in reducing nasal clinical reaction of EHV-1 infection in an animal. Alternatively, the invention is directed to a method to reduce nasal clinical reaction of EHV-1 infection comprising administering an immunogenic composition comprising an antigen of Equine Herpes Virus type 1 (EHV-1) and an antigen of Equine Herpes Virus type 4 (EHV-4) to an animal.

The reduction in clinical reaction is an improvement in comparison to animals of a non-treated control group of the same species, or to animals of the same species that are treated with an immunogenic composition comprising of EHV-1 but not EHV-4.

The reduction of clinical reaction may be selected from the group consisting of a shorter duration of clinical reaction, an earlier end to clinical reaction, a later onset of clinical reaction, a lower clinical reaction score or a lower severity of clinical reaction, and combinations thereof. Suitably the reduction in clinical reaction is a reduction in total clinical score and/or a reduction in the severity of the clinical reaction.

Suitably, in the immunogenic composition and the use and method thereof according to the invention and/or any embodiment thereof the EHV-1 and EHV-4 are inactivated. Suitably, the EHV-1 and EHV-4 are inactivated by an inactivating agent. Suitably the EHV-1 and EHV-4 are inactivated by an inactivating agent selected from the group comprising of formaldehyde and bromoethylamine hydrobromide.

Suitably the amount of EHV-4 is higher than the amount of EHV-1 in the immunogenic composition and the use or method thereof according to the invention and/or any embodiment thereof. Suitable ratios according to the invention and/or any embodiment thereof are ratios wherein EHV-1:EHV-4 is at least 1:1.1, at least 1:1.2, at least 1:1.3, at least 1:1.5, at least 1:1.7, at least 1:2. Suitable ratios according to the invention and/or any embodiment thereof are ratios wherein EHV-1:EHV-4 is 1:1.1 to 1:10, or 1:1.2 to 1:9, or 1:1.3 to 1:8, or 1:1.4 to 1:1:7, or 1:1.5 to 1:6, or 1:1.7 to 1:5, or 1:2 to 1:4.

A suitable dose of EHV-1 in the immunogenic composition and use thereof according to the invention and/or any embodiment thereof is between 500 to 10.000 EU. A suitable dose of EHV-4 in the immunogenic composition and use thereof according to the invention and/or any embodiment thereof is between 3000 to 40.000 EU. A suitable volume of a dose in the immunogenic composition and use thereof according to the invention and/or any embodiment thereof is between 0.1 to 2.5 mL.

Suitably, the immunogenic composition and use or method thereof according to the invention and/or any embodiment thereof further comprises a pharmaceutically acceptable carrier. Suitable pharmaceutically acceptable carrier in the immunogenic composition and use thereof according to the invention and/or any embodiment thereof may be selected from the group comprising, a diluent, an adjuvant, a stabiliser and a preservative.

Suitably, the immunogenic composition and use or method according to the invention and/or any embodiment thereof comprises an adjuvant. Suitably, the immunogenic composition according to the invention and/or any embodiment thereof comprises a saponin. Suitably, the immunogenic composition and use thereof according to the invention and/or any embodiment thereof further comprises an ingredient selected from the group consisting of buffer, medium, and salt.

Suitably, the use or method according to the invention and/or any embodiment thereof comprises administering the immunogenic composition according to the invention and/or any embodiment thereof to an animal in need thereof. Suitably the animal is an equine, preferably a horse. Suitably, the animal is a horse or foal of at least 3 month of age. Suitably, the animal is a pregnant mare.

Suitably, the use or method according to the invention and/or any embodiment thereof comprises administering the immunogenic composition according to the invention and/or any embodiment thereof in at least one dose. Suitably, the use or method according to the invention and/or any embodiment thereof comprises at least two doses. Suitably, the at least two doses are administered at least 15 days apart.

Suitably, the use or method according to the invention and/or any embodiment thereof comprises administering the immunogenic composition according to the invention and/or any embodiment thereof in at least two doses and then the at least two doses are followed by administration of a further dose, administered 3 to 24 month after the second dose. Suitably the at least two doses are followed by administration of further doses every 3 to 24 month.

In an embodiment of the invention and/or embodiments thereof, the animal is a pregnant mare and the animal is administered at least two doses. Suitably, the animal is a pregnant mare and the animal is administered at least three doses. Suitably, the animal is a pregnant mare and the animal is administered during month 5, 7, and/or 9 of gestation.

Suitably, the use or method according to the invention and/or any embodiment thereof comprises administering the immunogenic composition according to the invention and/or any embodiment thereof intramuscular or intranasal.

### DETAILED DESCRIPTION

### Legend to the Figures

FIG. 1. Clinical score (±s.e.m.) after challenge with EHV-1 strain AB-4.

FIG. 2. Body temperature (±s.e.m.) after challenge with EHV-1 strain AB-4.

FIG. 3. Total clinical score (clinical score+temperature score) (±s.e.m.) after challenge with EHV-1 strain AB-4.

FIG. 4: EHV-1 viremia score after challenge with EHV-1 strain AB-4.

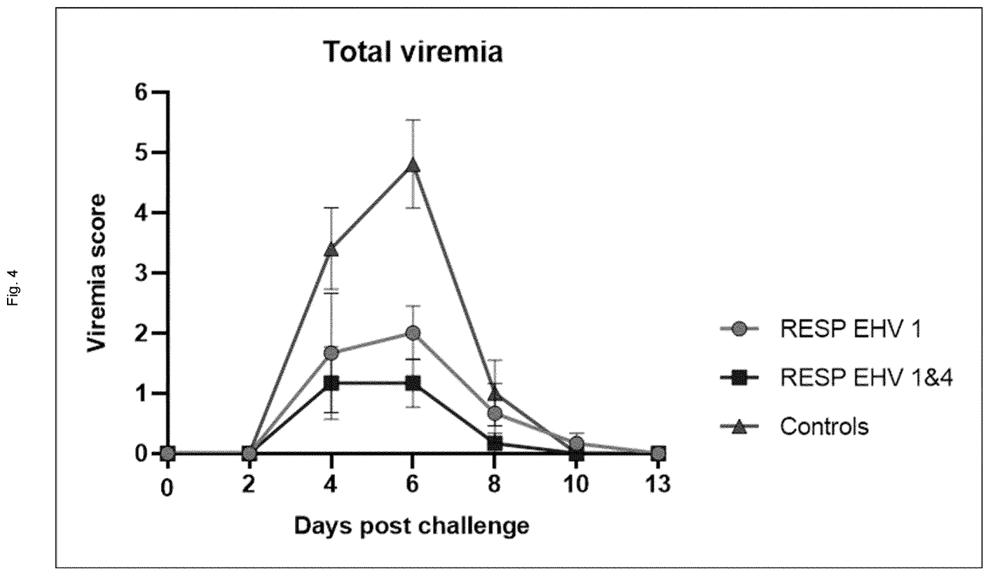

FIG. 5: Nasal clinical score after challenge with EHV-1 strain AB-4.

The term “EHV” as used herein refers to all equine herpes viruses such as species EHV-1 and EHV-4 within the family Alphaherpesvirinae. The terms EH-virus, EH virus EHV virus, EHV-virus and EHV all relate to equine herpes viruses.

The term “immunogenic composition” as used herein refers to a pharmaceutical composition comprising at least one immunologically active component that induces an immunological response in an animal and a pharmaceutically acceptable carrier. An immunogenic composition may also be referred to as a vaccine in the present specification. A vaccine may additionally comprise further components typical to pharmaceutical compositions. An immunogenic composition and a vaccine may be used interchangeable in the present specification. Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the composition or vaccine of interest. Suitably, the host will display either a therapeutic or protective immunological response such that resistance to new infection will be enhanced and/or the clinical severity of the disease reduced. Such protection will be demonstrated by either a reduction or lack of clinical signs normally displayed by an infected host, a quicker recovery time and/or a lowered duration or viral titer in the tissues or body fluids or excretions of the infected host. In particular, in the present invention, the protection is demonstrated by a reduction in viremia,, a lower virus load, and combinations thereof. In addition, the protection is demonstrated in the present invention by a reduction in overall clinical reaction and in particular in a reduction in nasal clinical reaction.

The immunologically active component of a/the immunogenic composition may comprise complete virus particles in either their original form or as attenuated particles in a so called modified live vaccine (MLV) or particles inactivated by appropriate methods in a so called killed vaccine (KV) or inactivated vaccine (inac or IV). In another form the immunologically active component of an/the immunogenic composition may comprise appropriate elements of said organisms (subunit vaccines) whereby these elements are generated either by destroying the whole particle or the growth cultures containing such particles and optionally subsequent purification steps yielding the desired structure(s), or by synthetic processes including an appropriate manipulation by use of a suitable system based on, for example, bacteria, insects, mammalian or other species plus optionally subsequent isolation and purification procedures, or by induction of said synthetic processes in the animal needing a vaccine by direct incorporation of genetic material using suitable pharmaceutical compositions (polynucleotide vaccination).

As used herein, “a pharmaceutically acceptable carrier” or “pharmaceutical carrier” includes any and all excipients, solvents, growth media, dispersion media, coatings, adjuvants, stabilizing agents, diluents, 30 preservatives, inactivating agents, antimicrobial, antibacterial and antifungal agents, isotonic agents, adsorption delaying agents, and the like. Such ingredients include those that are safe and appropriate for use in veterinary applications. Suitably, stabilizing agents for use in the present invention include stabilizers for lyophilization or freeze-drying.

“Diluents” may include water, saline, dextrose, ethanol, glycerol, and the like. Isotonic agents may include sodium chloride, dextrose, mannitol, sorbitol, and lactose, among others. Stabilizers may include albumin and alkali salts of ethylenediaminetetraacetic acid, among others.

hank ydjuvants” as used herein, can include aluminum hydroxide and aluminum phosphate, saponins e.g., Quil A, QS-21 (Cambridge Biotech Inc., Cambridge MA), GPI-0100, (Galenica Pharmaceuticals, Inc., Birmingham, AL), non-metabolizable oil, mineral and/or plant/vegetable and/or animal oils, polymers, carbomers, surfactants, natural organic compounds, plant extracts, carbohydrates, cholesterol, lipids, water-in-oil emulsion, oil-in-water emulsion, water-in-oil-in-water emulsion, HRA-3 (acrylic acid saccharide cross-linked polymer), HRA-3 with cottonseed oil (CSO), or preferably HRA-5 (acrylic acid polyol cross-linked polymer). The Emulsion can be based in particular on light liquid paraffin oil (European Pharmacopeia type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. Anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.) John Wiley and Sons, NY, pp 51-94 (1995) and Todd et al., Vaccine 15:564-570 (1997). Suitably, the adjuvant is at a concentration of about 0.01 to about 50%, or at a concentration of about 2% to 30%, or at a concentration of about 5% to about 25%, or at a concentration of about 7% to about 22%, or at a concentration of about 10% to about 20% by volume of the final product.

TCID50″ refers to tissue culture infective dose infecting 50% of cells in a culture inoculated with the virus.

For purposes of the present invention the terms “strain” and “isolate” have the same meaning and are used interchangeably.

“Clinical signs” or “clinical symptoms or clinical reactions” for Equine Herpes virus, for purposes of this invention include, but are not limited to, abortion, neurological deficiencies, respiratory disease, reproductive system deficiencies and failure, and symptoms relating to the central nervous system. Additionally, clinical symptoms of EHV-1 include, but are not limited to, the phenomenon of foals infected with EHV-1, exhibiting respiratory complications, passing the virus to the older members of the herd who then exhibit reproductive deficiencies, including abortion, and neurological deficiencies, normally exhibited in the central nervous system. Clinical signs also include viremia, duration of viremia, virus load, virus in the bloodstream, or virus in leukocytes. Suitably, the following clinical reactions are measured:
general health: mailaise or depression with or without normal appetite; anorexia, dehydration, unable to stand, diarrhoea, colic and dead;
Respiratory: Hyperpnoea, Dyspnoea and frequency of cough Ocular: Lachrymation, degree of mucopurulent discharge, degree of conjunctivitis
Nasal: degree of nasal discharge and mucopurulent and frequency of sneezing.
Neurological: Droopy lower lip, Oedema in one or more legs, Cocked head, Shuffling gait, Ataxia, Tail and anus tonus, and full bladder/incontinence

Suitably the clinical symptoms are converted to a clinical score which is a numerical score. In this way clinical reactions can quantitively be compared. Suitably the clinical observations are scored according to table 1 herein.

“Reduction of the incidence and/or severity of clinical signs” or “reduction in the incidence and/or severity of clinical symptoms,” as referred to herein, means reducing the number of infected animals in a group, reducing or eliminating the number of animals exhibiting clinical signs of infection, or reducing the severity of any clinical signs that are present in the animals, in comparison to wild-type infection. For example, such clinical signs included temperature, general health score, respiratory clinical reaction, nasal clinical reaction ocular clinical reaction, and neurological clinical reactions. Suitably, these are reduced in animals receiving the composition of the present invention by at least 10% in comparison to animals not receiving the vaccination which may become infected.

“Duration of Immunity,” as used herein, refers to the minimum time, such as number of days during which an animal produces an immunogenic response such that the animal will be relatively immune from contracting a virus and/or benefit from reduction of incidence and/or severity of clinical signs and or viremia, as described herein.

The term “inactivated” and “inactivated virus” refers to a previously virulent virus that has been treated to inactivate, kill, or otherwise modify the virus to substantially eliminate its virulent properties while retaining its immunogenicity. Suitably, the inactivated viruses disclosed herein are inactivated by treatment with an inactivating agent, such as heat or a chemical. Suitable inactivating agents include beta-propiolactone, binary ethyleneimine, glutaraldenyde, and formaldehyde. Suitably, the inactivating agent is formaldehyde or bromethylamide hybromide (BEA).

The invention provides an immunogenic composition comprising an antigen of Equine Herpes Virus type 1 (EHV-1) and antigen of Equine Herpes Virus type 4 (EHV-4). It was found that the immunogenic composition provided a reduction in viremia and a reduction in clinical reaction after infection with EHV-1 in animals. The viremia and clinical reaction were reduced when compared to animals that were not vaccinated but were also reduced when compared to animals that were vaccinated with EHV-1 but not EHV-4. Surprisingly, the amount of EHV-1 antigen in the immunogenic composition comprising EHV-1 antigen and EHV-4 antigen was lower than the amount of EHV-1 antigen in the vaccine comprising EHV-1 but not EHV-4. Even more surprisingly the total amount of antigen (i.e. EHV-1+EHV-4) in in the immunogenic composition comprising EHV-1 antigen and EHV-4 antigen was lower than the amount of EHV-1 antigen in the vaccine comprising EHV-1 but not EHV-4.

Suitably, the EHV-1 and the EHV-4 antigen in the immunogenic composition and use or method thereof of the present invention and/or embodiments thereof are combined in ratios of from about 1:10 to about 10:1. Suitable ratios of EHV 1:EHV-4 are between 1:10 to 1:1, suitably between 1:6 to 1:2, more suitably between 1:5 and 1:3, more suitably between 1:4 and 1:3.

In a preferred embodiment of the immunogenic composition and use or method thereof of the present invention and/or embodiments thereof, the amount of EHV-4 antigen is higher than the amount of EHV-1 antigen. Suitable ratios of EHV-1:EHV-4 antigen are between 1:10 to 1:1.1, between 1:9 to 1:1.5, between 1:8 to 1:2, between 1:7 to 1:2.5, between 1:6 to 1:3, between 1:5 to and 1:3.5. Suitably the ratio of EHV-1:EHV-4 is at least 1:1.1, suitably at least 1:1.2, suitably at least 1:1.5, suitably at least 1:2, suitably at least 1:2.5, suitably at least 1:3. Suitably the amount of EHV-4 antigen is at least 5%, at least 10%, at least 15%, at least 20%, at least 25%, at least 30%, at least 40%, at least 50%, at least 60%, at least 70%, at least 80%, at least 90%, at least 100% higher than the amount of EHV-1 antigen. Suitably, the amount of EHV-4 antigen is between 10% and 1000% higher than the amount of EHV-1 antigen, or the amount of EHV-4 antigen, is between 15% and 900% higher, between 20% and 800% higher, between 25% and 700% higher, between 30% and 600% higher, between 35% and 500% higher, between 40% and 450% higher, between 45% and 400% higher, or between 50% and 350% higher, or between 55% and 300% higher, or between 60% and 250% higher, or between 65% and 200% higher, or between 70% and 150% higher, or between 75% and 100% higher than the amount of EHV-1 antigen.

Equine Herpes Virus (“EHV”) strains useful in the vaccine or immunogenic composition of the present invention can be any strain or isolate. Representative strains include EHV-1 strain 438/77 and EHV-4 strain 405/76 viable strains of which have been deposited with the American Type Culture Collection, Rockville, Md. with accession numbers VR2229 and VR2230, EHV-1, EHV-4, strains deposited with the ATCC under accession Nos. PTA-9525 And PTA-9526, EHV Subtype 1, deposited as ATCC Accession NO: PTA-9525, and EHV Subtype 4, deposited As ATCC Accession NO: PTA-9526 deposited with the ATCC at 10801 University Boulevard, Manassas, VA, 20110-2209 on Sep. 23, 2008, under the provisions of the Budapest Treaty, EHV-4 Strain 2252, EHV-1 strain RacH, EHV-1.

Suitably the EHV useful in the vaccine or immunogenic composition of the present invention is not an EHV mutant. Suitably the EHV useful in the vaccine or immunogenic composition of the present invention is not an EHV mutant that is gM negative. Suitably the EHV useful in the vaccine or immunogenic composition of the present invention is not an EHV mutant wherein the gene encoding the gM protein is absent. Suitably the EHV useful in the vaccine or immunogenic composition of the present invention is not an EHV mutant as described by WO2004/009802.

Suitably the EHV useful in the vaccine or immunogenic composition of the present invention is an EHV mutant. Suitably the EHV useful in the vaccine or immunogenic composition of the present invention is an EHV mutant that is gM negative. Suitably the EHV useful in the vaccine or immunogenic composition of the present invention is an EHV mutant as described by WO2004/009802. Suitably the EHV useful in the vaccine or immunogenic composition of the present invention is an EHV wherein the gene encoding the gM protein is present.

Suitably the EHV useful in the vaccine or immunogenic composition of the present invention is not a live recombinant EHV virus. Suitably the EHV useful in the vaccine or immunogenic composition of the present invention is not a live recombinant EHV virus wherein US2 gene has been deleted. Suitably the EHV useful in the vaccine or immunogenic composition of the present invention is a live recombinant EHV virus wherein US2 gene is absent. Suitably the EHV useful in the vaccine or immunogenic composition of the present invention is not a live recombinant EHV virus as described in WO94/03628.

Suitably the EHV useful in the vaccine or immunogenic composition of the present invention is a live recombinant EHV virus. Suitably the EHV useful in the vaccine or immunogenic composition of the present invention is a live recombinant EHV virus wherein US2 gene has been deleted. Suitably the EHV useful in the vaccine or immunogenic composition of the present invention is a live recombinant EHV virus wherein US2 gene is present. Suitably the EHV useful in the vaccine or immunogenic composition of the present invention is a live recombinant EHV virus as described in WO94/03628.

Suitably the EHV useful in the vaccine or immunogenic composition of the present invention is not a recombinant equine herpesvirus comprising viral DNA from a species of equine herpesvirus and foreign DNA. Suitably the EHV useful in the vaccine or immunogenic composition of the present invention is not a recombinant equine herpesvirus comprising viral DNA from a species of equine herpesvirus and foreign DNA the foreign DNA being inserted into the equine herpesviral DNA at a site which is not essential for replication of the equine herpesvirus. Suitably the EHV useful in the vaccine or immunogenic composition of the present invention is not a live recombinant EHV virus as described in WO95/22607.

Suitably the EHV useful in the vaccine or immunogenic composition of the present invention is a recombinant equine herpesvirus comprising viral DNA from a species of equine herpesvirus and foreign DNA. Suitably the EHV useful in the vaccine or immunogenic composition of the present invention is a recombinant equine herpesvirus comprising viral DNA from a species of equine herpesvirus and foreign DNA the foreign DNA being inserted into the equine herpes-viral DNA at a site which is not essential for replication of the equine herpesvirus. Suitably the EHV useful in the vaccine or immunogenic composition of the present invention is a live recombinant EHV virus as described in WO95/22607.

The amount of EHV-1 and EHV-4 antigen in the immunogenic composition and use or method thereof of the present invention and/or embodiments thereof in a dose is between 500 and 40.000 EU. For EHV-1 antigen the amount in a dose is between 250 and 15.000. Suitably the amount in a dose of EHV-1 antigen is between 500 and 10.000 EU. More suitably the amount of EHV-1 antigen in a dose is between 500 and 5.000 EU, or between 600 and 3.000 EU, or between 700 and 2.500 EU, or between 800 and 2.000 EU, or between 900 and 1.750 EU or between 1.000 and 1.500 EU. Suitably, the amount of EHV-1 antigen in a dose is about 500, 600, 700, 800, 900, 1.000, 1.100, 1.200, 1.300, 1.400, 1.500, 1.600, 1.700, 1.800, 1.900, or 2.000 EU. Suitably the amount of EHV-1 antigen in a dose is below 10.000 EU, more suitably below 7.000 EU, more suitably below 6.000 EU, more suitably below 5.000 EU, more suitably below 3.000 EU, more suitably below 2.000 EU, more suitably below 1.900 EU, more suitably below 1.800 EU, more suitably below 1.700 EU, more suitably below 1.600 EU, more suitably below 1.500 EU, more suitably below 1.400 EU, more suitably below 1.300 EU, more suitably below 1.200 EU, more suitably below 1.100 EU, more suitably below 1.000 EU, more suitably below 900 EU, more suitably below 850 EU, more suitably below 800 EU, more suitably below 750 EU, more suitably below 700 EU, more suitably below 650 EU, more suitably below 600 EU, more suitably below 550 EU, more suitably below 500 EU.

For EHV-4 antigen the amount in a dose is suitably between 1.500 and 40.000 EU. More suitably the amount in a dose of EHV-4 antigen is between 2.000 and 35.000 EU, or more suitably between 2.500 and 30.000 EU, or more suitably between 2.000 and 25.000 EU, or between 2.500 and 20.000 EU, or between 3.000 and 17.500 EU, or between 3.500 and 15.000 EU, or between 4.000 and 12.500 EU, or between 4.500 and 10.000 EU, or between 5.000 and 7.500 EU. Suitably the amount in a dose of EHV-4 antigen is between 2.000 and 7.000 such as 2.000, 2.200, 2.400, 2.600, 2.800 3.000, 3.200, 3.400, 3.600, 3.800, 4.000, 4.200, 4.400, 4.600, 4.800, 5.000, 5.200, 5.400, 5.600, 5.800, 6.000, 6.200, 6.400, 6.600, 6.800, 7.000 EU.

Suitably the amount in a dose of EHV-1 antigen is between 800 and 1.500 EU such as 850, 900, 950, 1.000, 1.050, 1.100, 1.150, 1.200, 1.250, 1.300, 1.350, 1.400, 1.450, 1500 EU. Suitably the dose of EHV-4 antigen is between 2.800 and 4.500 such as 2.900, 3.000, 3.100, 3.200, 3.300, 3.400, 3.500, 3.600, 3.700, 3.800, 3.900, 4.000, 4.100, 4.200, 4.300, 4.400, or 4.500 EU.

Suitably the dose of EHV-1 antigen is at least 800 such as at least 850, at least 900, at least 950, at least 1.000, at least 1.050, at least 1.100, at least 1.150, at least 1.200, at least 1.250, at least 1.300, at least 1.350, at least 1.400, at least 1.450, or at least 1.500 EU. Suitably the dose of EHV-4 antigen is at least 2.800 such as at least 2.900, at least 3.000, at least 3.100, at least 3.200, at least 3.300, at least 3.400, at least 3.500, at least 3.600, at least 3.700, at least 3.800, at least 3.900, at least 4.000, at least 4.100, at least 4.200, at least 4.300, at least 4.400, or at least 4.500 EU.

EU are ELISA units and can be measured in any suitable way known to a skilled person. An ELISA may be used to quantify the amount of EHV-1 and/or EHV-4 in fluids. For examples, the wells of a polystyrene microtitre plate are coated with a (coating) monoclonal antibody which binds EHV-1 and/or EHV-4. Serial dilutions of antigen captured by the plate bound antibody are detected by sequentially adding biotin-labelled EHV-1 or EHV-4 monoclonal antibody and a horseradish peroxidase conjugate. The amount of peroxidase conjugate bound is correlated with the quantity of antigen captured and is determined by the enzymatic conversion of a colorless substrate into a blue reaction product. The reaction is stopped e.g. by adding H2SO4 and the color changes e.g. from blue to yellow. The amount of antigen is calculated using a reference curve that is run in parallel on each plate and is expressed as antigen units per ml (AU/ml).

Suitable carriers for the EHV immunogenic composition and use or method thereof according to the present invention and/or embodiments thereof are well known in the art and include diluent, adjuvant, antimicrobial agent, stabilizer, preservative, inactivating agent, or combination thereof. Suitably diluents may comprise distilled water, salts, medium ingredient, sterile water, buffer, stabilizer, preservative, bactericides, antibiotic, and chemicals to assist in dissolving. The carrier may comprise, NaCl, sodium carbonate, thiomersal, buffered saline, sucrose, disodium adipate, disodium hydrogen phosphate, sodium-dihydrogen phosphate dihydrate,

Stabilisers may include sugars such as lactose and sucrose, amino acids such as glycine and monosodium glutamate (salts of amino acids), proteins such as (recombinant) albumin derived from baker's yeast or fetal bovine (cow) serum and gelatin, such as hydrolysed collagen.

Suitably the vaccine or immunogenic composition of the invention and/or any embodiment thereof comprises an adjuvant. Suitably adjuvants in vaccine or immunogenic compositions according to the invention and/or any embodiment thereof may include aluminum hydroxide, aluminum phosphate, saponins, saponins complexed with cholesterol, saponin complexed with cholesterol and lipids, non-metabolizable oil, mineral and/or plant/vegetable and/or animal oils, polymers, carbomers, surfactants, natural organic compounds, plant extracts, carbohydrates, cholesterol, lipids, water-in-oil emulsion, oil-in-water emulsion, water-in-oil-in-water emulsion, acylic acid polymer, acrylic acid saccharide cross-linked polymer, acrylic acid polyol cross-linked polymer. Suitably adjuvants in vaccine or immunogenic compositions according to the invention and/or any embodiment thereof may include aluminum hydroxide, aluminum phosphate, saponins, saponins complexed with cholesterol, saponin complexed with cholesterol and lipids, mineral oils, polymers, carbomers, surfactants, natural organic compounds, carbohydrates, cholesterol, lipids, water-in-oil emulsion, oil-in-water emulsion, water-in-oil-in-water emulsion, acylic acid polymer. Suitably adjuvants in vaccine or immunogenic compositions according to the invention and/or any embodiment thereof may include aluminum hydroxide, aluminum phosphate, saponins, Quil A, QS-21, GPI-0100, non-metabolizable oil, mineral and/or plant/vegetable and/or animal oils, polymers, carbomers, surfactants, natural organic compounds, plant extracts, carbohydrates, cholesterol, lipids, water-in-oil emulsion, oil-in-water emulsion, water-in-oil-in-water emulsion, HRA-3 (acrylic acid saccharide cross-linked polymer), HRA-3 with cottonseed oil (CSO), or HRA-5 (acrylic acid polyol cross-linked polymer), ISCOMATRIX. Suitably the adjuvant is a saponin, acrylic acid polymers, and/or aluminum hydroxide. Suitably saponins are Quil A, ISCOM, QS21, ISCOPREP 703, ISCOMATRIX, AS series, ISCOPREP saponin, GPI0100, AbISCO, Matrix M, Matrix C, Posintro. Suitably acrylic acid polymers are Carbopol such as HRA-3, HRA-5, Carbopol 940, Carbopol 941, Carbopol 934, Carbopol 971, Havlogen, CARBIGEN. Suitable adjuvants are adjuvants based on saponin, more suitably adjuvants based on quillaia saponin. Examples of saponin based adjuvants are Quil A, ISCOM, QS21, ISCOMATRIX, ISCOPREP 703, AS series, GPI0100, AbISCO, Posintro. Suitable adjuvants are saponin based complexed with cholesterol. Suitable cholesterol complexed saponins are AS01, AS15, AS02, ISCOM, ISCOMATRIX, Matrix-M, AbISCO.

Suitably, the adjuvant is at a concentration of about 0.01 to about 50%, or at a concentration of about 2% to 45%, or at a concentration of about 5% to about 40%, or at a concentration of about 7% to about 35%, or at a concentration of about 10% to about 30% by volume of the final product. Suitably, the adjuvant is at a concentration of about 0.01 to about 10%, or at a concentration of about 0.02 to about 5%, or at a concentration of about 0.05 to about 2.5%, or at a concentration of about 0.1 to about 2%, or at a concentration of about 0.15 to about 1.5%, or at a concentration of about 0.2 to about 1.2%, or at a concentration of about 0.3 to about 1%, or at a concentration of about 0.4 to about 0.8%, or at a concentration of about 0.5 to about 0.7%,

In the composition of the present invention, EHV-1 and EHV-4 antigen are preferably inactivated EHV-1 and inactivated EHV-4. Suitable inactivating agents include beta-propiolactone, binary ethyleneimine (BEI), glutaraldehyde, bromoethylamine hydrobromide (BEA), aziridine compounds, and formaldehyde. Suitably, the inactivating agent is formaldehyde or binary ethyleneimine. Methods of inactivation of virus are well known to a skilled person. For example inactivation of the viruses may be achieved by the use of 0.2% beta propiolactone for 24 hours at 4⋅C, or incubated in BEI concentrations of 0.4 mM, 0.8 mM, 1.2 mM, 1.6 mM or 2.0 mM at 37° C. with continuous stirring for 10-24 h.

The immunogenic composition of the present invention and/or embodiments thereof provides a duration of immunity of at least 6 months against EHV-1 and/or EHV-4 infection. The immunogenic composition of the present invention and/or embodiments thereof provides a duration of immunity against EHV-1 and/or EHV-4 infection. Preferably, the duration of immunity against EHV-1 and/or EHV-4 infection is at least 1 month, more preferably, the duration of immunity is at least 2 months, still more preferably, the duration of immunity is at least 3 months, even more preferably, the duration of immunity is at least 4-24 months, still more preferably, the duration of immunity is at least 6-24 months, even more preferably, the duration of immunity is at least 7-24 months, still more preferably, the duration of immunity is at least 8-24 months, even more preferably, the duration of immunity is at least 9-24 months, still more preferably, the duration of immunity is at least 10-24 months, and most preferably, the duration of immunity is at least 12-24 months.

The method of preparing each vaccine or immunogenic composition according to the invention and/or embodiments thereof, includes the steps of
growing the selected strain of EHV-4 in an equine dermis cell or other appropriate cell line;
growing the selected strain of EHV-1 in an equine dermis cell line, or other appropriate cell;
inactivating EHV-4
inactivating EHV-1 and
combining the inactivated EHV-1 and inactivated EHV-4 with a suitable diluent, adjuvant and/or preservative.

The invention provides an immunogenic composition that can be used to reduce virema in an EHV-infected animal. The invention relates to a method of reducing virema in an EHV-infected animal wherein the immunogenic composition according to the invention is administered to the animal at a suitable dose.

Suitably, viremia is reduced by at least 10% in comparison to animals not receiving the vaccination, or by at least 20%, by at least 30%, by at least 40% and even by at least 50%, or by at least 60% or by at least 70%, or by at least 80%. Suitably, the height of the peak of viremia is reduced by at least 10% in comparison to animals not receiving the vaccination, or by at least 20%, by at least 30%, by at least 40% and even by at least 50%, or by at least 60% or by at least 70%, or by at least 80% in comparison to animals not receiving the vaccination.

Suitably, viremia is reduced by at least 10% in comparison to animals having received a vaccination with EHV-1 but not EHV-4, or by at least 20%, by at least 30%, by at least 40% and even by at least 50%. Suitably, the height of the peak of viremia is reduced by at least 10% in comparison to animals having received a vaccination with EHV-1 but not EHV-4, or by at least 20%, by at least 30%, by at least 40% and even by at least 50%.

Presence and quantification of EHV in animals can be measured by many methods known to the skilled person. Isolated (blood) cells can be used for cell culture to determine if EHV is present or not. Dilutions of the isolated cells provide an indication of the amount of EHV in the cells. In addition, measurement of EHV in an animal can be carried on isolated (blood) cells and tissue using PCR, see e.g. Azab et al (Veterinary Medicine and Science (2019), 5, pp. 361-371); Hussey et al (Vet Diagn Invest 18:335-342 (2006)); Pusterla et al. (J Vet Diagn Invest 17:578-581 (2005)).

The immunogenic composition is preferably administered to an Equidae, preferably a horse.

The dosing scheme may include administration of the immunogenic composition in one or more doses. Suitably, the immunogenic compositions of the present invention are safe for use in foals or horses 3 months of age or older, preferably foals or horses of 4 months of age or older, preferably foals or horses of 5 months of age or older, preferably foals or horses of 6 months of age or older.

In a preferred embodiment, the immunogenic composition is administered as a single dose. In another embodiment, the immunogenic composition is administered with a first dose being administered prior to the administration of a second (booster) dose. Preferably, the second dose is administered at least about 15 days after the first dose. More preferably, the second dose is administered between 15 days and 49 days after the first dose, or between 17 days and 42 days after the first dose, or between 20 days and 37 days after the first dose, or between 22 days and 35 days after the first dose, or between 25 days and 32 days after the first dose, or between 27 days and 30 days after the first dose. Also preferred, the second dose is administered at least 17 days after the first dose. Still more preferably, the second dose is administered between 17 and 25 days after the first dose. Even more preferably, the second dose is administered at least 19 days after the first dose. Still more preferably, the second dose is administered between 19 and 23 days After the first dose. Most preferably the second dose is administered at least 21 days after the first dose. In a preferred embodiment, both the first and second doses of the immunogenic Suitably, the composition are in the same amount. Preferably, each dose is in the preferred amounts specified above.

In addition to the first and second dose regimen, an alternate embodiment comprises further subsequent doses. For example, a third, fourth, or fifth dose could be administered in these embodiments. Preferably, subsequent third, fourth, and fifth dose regimens are administered in the same amount as the first dose, with the time frame between the doses being consistent with the timing between the first and second doses mentioned above, although the timing may also vary. Suitably doses regimes after first 2 doses may comprise an additional vaccination every 3 month, or every 6 months, or every 9 months, or every 12 months, or every 15 months, or every 18 months, or every 21 months, or every 24 months.

Suitably, pregnant mares are vaccinated every two months throughout pregnancy. Suitable doses regimes for pregnant mares are vaccinations during the 5^{th}, 7^{th}, and 9^{th} month of gestation.

Preferably, the dose has a total volume between 0.5 ml and 2.5 ml, more preferably between 0.6 ml and 2.0 ml, even more preferably between 0.7 ml and 1.75 ml, still more preferably between 0.8 ml and 1.5 ml, even more preferably between 0.9 ml and 1.25 ml, with a dose of 1.0 ml being the most preferred.

The compositions as disclosed herein can be administered in any conventional manner. Examples of administration methods include any that afford access by cells of the immune system to the immunogenic composition including oral, transdermal/intradermal, Intravenous, subcutaneous, intramuscular, intraocular, intraperitoneal, intrarectal, intravaginal, Intranasal, intragastrical, intratracheal, intrapulmonarial, or any combination thereof. In a preferred embodiment, the vaccine or immunogenic composition according to the invention and/or any embodiment thereof is administered parenterally, preferably intranasally, subcutaneously, or intramuscularly, and in the most preferred embodiment the vaccine is administered intramuscularly.

The immunogenic compositions of the invention and/or any embodiment thereof may include, in addition to EHV-1 and EHV-4 antigen at least one further antigen. The further antigen may be selected from the group consisting of inactivated or live, BEI concentrations of 0.4 mM, 0.8 mM, 1.2 mM, 1.6 mM and 2.0 mM attenuated virus or bacterium. Suitably the further antigen is an antigen selected from the group consisting of one or more strains selected from the group consisting of EHV-5, EHV-2, West Nile Virus, Eastern Equine Encephalomyelitis Virus, Western Equine Encephalomyelitis Virus, Venezuelan Equine Encephalomyelitis Virus, Equine Influenza virus, Rhodococcus equi, Streptococcus equi, and Tetanus Toxoid, and combinations thereof. The amount of further equine antigen is the amount that is usually present in a vaccine comprising such additional equine antigen.

The present invention also provides a method for the treatment or prophylaxis of EHV-1, comprising the step of administering an effective amount of an immunogenic composition of the present invention to an animal in need of such treatment, wherein the treatment or prophylaxis results in reducing viremia and/or reducing the severity of viremia. Moreover, the immunogenic composition described herein reduces the overall virus load including a later onset, a shorter duration, an earlier end of viremia, and a reduced viral load.

Suitably, viremia is reduced by at least 10% in comparison to animals not receiving the vaccination, or by at least 20%, by at least 30%, by at least 40% by at least 50%, by at least 60%, by at least 70%, by at least 75%, by at least 80%, by at least 85%, by at least 90%, and even by at least 95%. Suitably viremia is reduced by at least 50% in comparison to animals not receiving the vaccination, or by at least 60%, by at least 70%, by at least 75% by at least 80%. Suitably, viremia is reduced by at least 10% in comparison to animals vaccinated with EHV-1 vaccine, or by at least 20%, by at least 30%, by at least 40% by at least 50%, by at least 60%, by at least 70%, by at least 75%, by at least 80%, by at least 85%, by at least 90%, and even by at least 95%. Suitably viremia is reduced by at least 20% in comparison to animals vaccinated with EHV-1 vaccine, or by at least 25%, by at least 30%, by at least 35%, or by at least 40%, or by at least 45%, or by at least 50%.

The present invention also provides a method of reducing the incidence or severity of clinical symptoms associated with EHV comprising the step of administering any one of the immunogenic compositions according to the invention to an animal.

Suitably, the reduction in clinical signs when compared to animals not receiving the immunogenic composition, is at least a 10% reduction in clinical signs.

The present invention provides a method of reducing the incidence and severity of clinical symptoms of EHV in a herd, comprising the step of administering any one of the immunogenic compositions according to the invention to an animal.

Suitably, the clinical symptoms are selected from the group consisting of respiratory disease, abortion, reproductive complications, neurological disease, central nervous system disease, and combinations thereof.

Suitably, the following clinical reactions are measured:
general health: mailaise or depression with or without normal appetite; anorexia, dehydration, unable to stand, diarrhoea, colic and dead;
Respiratory: Hyperpnoea, Dyspnoea and frequency of cough Ocular: Lachrymation, degree of mucopurulent discharge, degree of conjunctivitis
Nasal: degree of nasal discharge and mucopurulent and frequency of sneezing.
Neurological: Droopy lower lip, Oedema in one or more legs, Cocked head, Shuffling gait, Ataxia, Tail and anus tonus, and full bladder/incontinence

Suitably the clinical symptoms are converted to a clinical score which is a numerical score. In this way clinical reactions can quantitively be compared. Suitably the clinical observations are scored according to table 1 herein.

Suitably, the total clinical signs or reactions are reduced in animals receiving the composition of the present invention by at least 20%, or by at least 30%, or by at least 40%, or by at least 50%, or by at least 60% or by at least 70%, or by at least 80% in comparison to animals not receiving the vaccination. Suitably the severity of the total clinical signs or reaction is reduced by at least 20%, or by at least 30%, or by at least 40%, or by at least 50%, or by at least 60% or by at least 70%, or by at least 80% in comparison to animals not receiving the vaccination.

Suitably, the total clinical signs or reactions are reduced in animals receiving the composition of the present invention by at least 20%, or by at least 30%, or by at least 40%, or by at least 50%, or by at least 60% in comparison to animals not receiving the vaccination or receiving a vaccination with EHV-1 antigen but not EHV-4 antigen. Suitably the peak of the total clinical signs or reaction is reduced by at least 20%, or by at least 30%, or by at least 40%, or by at least 50%, or by at least 60% or by at least 70% in comparison to animals not receiving the vaccination or receiving a vaccination with EHV-1 antigen but not EHV-4 antigen.

The present invention covers the following embodiments. It is expressly intended that these embodiments can be combined, however for brevity of the description, the specific combinations are not explicitly described.

In a first embodiment, the invention is directed to an immunogenic composition comprising an antigen of Equine Herpes Virus type 1 (EHV-1) and an antigen of Equine Herpes Virus type 4 (EHV-4).

In another embodiment, the invention is directed to an immunogenic composition comprising an antigen of EHV-1 and an antigen of EHV-4 for use in reducing viremia in an animal.

In another embodiment, the invention is directed to a method of reducing viremia in an animal comprising administering an immunogenic composition comprising an antigen of EHV-1 and an antigen of EHV-4 to an animal.

In another embodiment, the reduction in viremia is an improvement in comparison to animals of a non-treated control group of the same species.

In another embodiment, the reduction in viremia is an improvement in comparison to animals of the same species that are treated with an immunogenic composition comprising of EHV-1 but not EHV 4.

In another embodiment, the reduction of viremia is selected from the group consisting of a lower virus load, a lower peak virus load, and combinations thereof.

In another embodiment, the invention is directed to an immunogenic composition comprising an antigen of EHV-1 and an antigen of EHV-4 for use in reducing clinical reaction to an infection of EHV-1 in an animal.

In another embodiment, the invention is directed to a method of reducing clinical reaction to an infection of EHV-1 in an animal comprising administering an immunogenic composition comprising an antigen of EHV-1 and an antigen of EHV-4 to an animal.

In another embodiment, the EHV-1 antigen and EHV-4 antigen are inactivated EHV-1 and inactivated EHV-4.

In another embodiment, the EHV-1 and EHV-4 are inactivated by an inactivating agent.

In another embodiment, the EHV-1 and EHV-4 are inactivated by an inactivating agent selected from the group comprising of formaldehyde and bromethylamide hybromide.

In another embodiment, the amount of EHV-4 antigen is higher than the amount of EHV-1 antigen.

In another embodiment, the ratio of EHV-1:EHV-4 antigen is 1:1.1 to 1:10.

In another embodiment, a dose of EHV-1 antigen is between 500 to 10.000 EU.

In another embodiment, a dose of EHV-4 antigen is between 2500 to 40.000 EU.

In another embodiment, the volume of a dose is between 0.1 to 2.5 mL.

In another embodiment, the immunogenic composition further comprises a pharmaceutically acceptable carrier.

In another embodiment, the immunogenic composition further comprises a pharmaceutically acceptable carrier selected from the group comprising, diluent, adjuvant, stabiliser, preservative.

In another embodiment, the immunogenic composition further comprises an adjuvant.

In another embodiment, the immunogenic composition further comprises a saponin.

In another embodiment, the immunogenic composition further comprises an ingredient selected from the group consisting of buffer, medium, and salt.

In another embodiment, the immunogenic composition is administered to an animal in need thereof.

In another embodiment, the animal is an equine, preferably a horse.

In another embodiment, the animal is a horse or foal of at least 3 month of age.

In another embodiment, the animal is a pregnant mare.

In another embodiment, the animal is administered with at least one dose of the immunogenic composition according to the invention and/or any embodiment thereof.

In another embodiment, the animal is administered with at least two doses of the immunogenic composition according to the invention and/or any embodiment thereof.

In another embodiment, the animal is administered with at least two doses wherein the two doses are administered at least 15 days apart.

In another embodiment, the animal is administered with at least two doses wherein the two doses are followed by administration of a further dose of the immunogenic composition according to the invention and/or any embodiment thereof, wherein the further dose is administered 3 to 24 month after the second dose.

In another embodiment, the animal is administered with at least two doses of the immunogenic composition according to the invention and/or any embodiment thereof wherein the two doses are followed by administration of further doses every 3 to 24 month.

In another embodiment, the animal is a pregnant mare and the animal is administered at least two doses of the immunogenic composition according to the invention and/or any embodiment thereof.

In another embodiment, the animal is a pregnant mare and the animal is administered at least three doses of the immunogenic composition according to the invention and/or any embodiment thereof.

In another embodiment, the animal is administered with the immunogenic composition according to the invention and/or any embodiment thereof during month 5, 7, and/or 9 of gestation.

In another embodiment, the administration of the immunogenic composition according to the invention and/or any embodiment thereof is intramuscular or intranasal.

The invention will now be further described by the following, non-limiting, examples.

### EXAMPLES

### Study design

Seventeen Shetland ponies were divided into 3 groups. On day 0 and 28, ponies of group 1 and 2 were vaccinated intramuscular (i.m.) with test vaccine according to table 1. Group 3 served as the negative control group. On day 49 all ponies were challenged intranasal (i.n.) with a virulent EHV-1 strain.

TABLE 1
Experimental design

| | | |
|---|---|---|
| No. | Vaccination inoculum | Challenge virus |

| Group | horses | Day 0 | Day 28 | Day 49 |
|---|---|---|---|---|
| 1 | 6 | EHV1 | EHV1 | EHV-1 |
| | | 2 mL i.m. | 2 mL i.m. | 1 mL i.n. |
| 2 | 6 | EHV1&4 | EHV1&4 | EHV-1 |
| | | 1 mL i.m. | 1 mL i.m. | 1 mL i.n. |
| 3 | 5 | — | — | EHV-1 |
| | | | | 1 mL i.n. |

EHV1 vaccine contained inactivated EHV-1 (RAC-H) 4165 EU/ml, and Matrix C in 2 mL
EHV1&4 vaccine contained inactivated EHV-1 991.6 EU/ml and 3583.5 EHV-4 EU/ml and Matrix C in 1 mL

Challenge material was a live EHV-1, strain AB4, 10^{6.74} TCID₅₀/ml. Challenge virus was applied undiluted, 10^{6.74} TCID₅₀/horse.

Matrix C is a ISCOMATRIX adjuvant. The ISCOMATRIX adjuvant contains purified fractions of Quillaia Saponaria extract (ISCOPREP saponin), cholesterol and phospholipid, which combine under controlled conditions to form cage-like structures, typically 40-50 nm in diameter (Morelli et al. Journal of Medical Microbiology (2012), 61, 935-943). For each vaccine 350 μg Matrix-C was added per dose.

TABLE 2
Composition vaccines

| vaccina- | | | | |
|---|---|---|---|---|
| tion | strain | amount | adjuvant | dose |
| EHV 1 | RAC-H | 4165 EU/ml | Matrix C | 2 mL |
| | | | 350 μg/dose | |
| EHV 1&4 | EHV1: RAC-H | EHV1: 991.6 EU/ml | Matrix C | 1 mL |
| | EHV4: 2252 | EHV4: 3583.5 EU/ml | 350 μg/dose | |

EHV-1 Strain RAC-H is inactivated by 10 mM bromoethylamine hydrobromide (BEA) for sixteen hours at 37° C. Acid that is formed is neutralized by addition of sodium hydroxide solution to maintain a pH of 7.4 (range 7.3-7.6). After the incubation period the BEA is neutralized by addition of 20 mM sodium thiosulphate at 37° C. during 2 hours. Thereafter the preparation is checked for complete inactivation of the virus.
EHV-4 Strain 2252 is inactivated by 10 mM bromethylamide hybromide (BEA) for sixteen hours at 37° C. Acid that is formed is neutralized by addition of sodium hydroxide solution to maintain a pH of 7.4 (range 7.3-7.6). After the incubation period the BEA is neutralized by addition of 20 mM sodium thiosulphate at 37° C. during 2 hours. Thereafter the preparation is checked for complete inactivation of the virus.

Only healthy horses were used for this study. Before start of the study animals were included based on a pre-screening for EHV and EIV antibody titres. Ponies were seronegative for EHV-1, EHV-4 and EIV.

Horses were vaccinated intramuscular (i.m.) in the neck on day 0 and 28. Horses were challenged intranasal (i.n.) on day 49 with 1 mL of the challenge material.

### Clinical Observations

Clinical observation were made based on general health and respiratory, ocular, nasal and neurological signs typical for EHV infection were scored according to the Elms-Gravely system. To each observation a score was assigned based on animal welfare according to table 3. Protection to clinical signs post challenge was assessed by analysing the observed clinical signs and measured body temperature. There were clinical signs of coughing, sneezing, dyspnoea and nasal and ocular discharge.

TABLE 3
Clinical reaction score chart

| | |
|---|---|
| Clinical reaction | Score |

GENERAL HEALTH

| | |
|---|---|
| No abnormality found | 0 |
| Malaise/depression/normal eating | 1 |
| Malaise/depression/reduced appetite | 2 |
| Anorexia | 4 |
| Dehydration | 2 |
| Down/unable to stand | 30 |
| Diarrhoea | 1 |
| Colic | 5 |
| Dead | 100 |

RESPIRATORY

| | |
|---|---|
| Hyperpnoea | 2 |
| Dyspnoea | 4 |
| Cough/2-5x in 10 minutes | 1 |
| Cough/6-20x in 10 minutes | 2 |
| Cough/>20x in 10 minutes | 3 |

OCULAR

| | |
|---|---|
| Lachrymation | 1 |
| Mild mucopurulent discharge | 2 |
| Marked mucopurulent discharge | 4 |
| Mild conjunctivitis | 2 |
| Marked conjunctivitis | 4 |

NASAL

| | |
|---|---|
| Nasal serous discharge | 1 |
| Mild nasal mucopurulent | 2 |
| Marked nasal mucopurulent | 4 |
| Sneeze/2-5x in 10 minutes | 1 |
| Sneeze/6-20x in 10 minutes | 2 |
| Sneeze/>20x in 10 minutes | 3 |

NEUROLOGICALY

| | |
|---|---|
| Droopy lower lip | 3 |
| Oedema in one or more legs | 3 |
| Cocked head | 3 |
| Shuffling gait | 3 |
| Ataxia | 3 |
| Tail tonus (during temperature measuring) | 3 |
| Anus tonus (during temperature measuring) | 3 |
| Full bladder/incontinence | 3 |

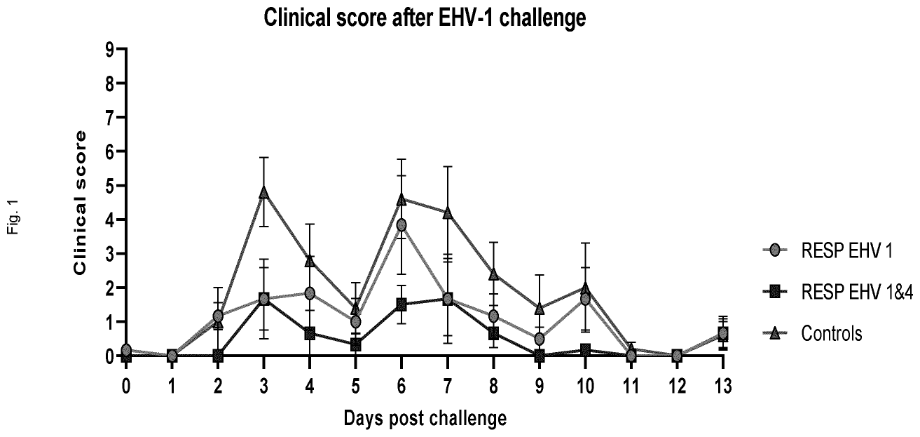

Clinical signs were observed in the control group from 2-10 days post challenge (d.p.c.) peaking on 3 and 6 d.p.c., with an average clinical score of 4.8 and 4.6 respectively (FIG. 1). In the EHV 1 group clinical signs were observed from 2-10 d.p.c. peaking on 3, 6 and 10 d.p.c., with an average clinical score of 1.8, 3.8 and 1.7 respectively. The clinical score was lower at most time points compared to the controls. In the EHV 1&4 group clinical signs were observed from 3-8 d.p.c. peaking on 3 and 7 d.p.c., with an average clinical score of 1.7 and 1.7 respectively. The clinical score was clearly lower compared to the controls and overall lower compared to the EHV 1 group. The clinical score in both vaccinated groups was clearly lower compared to the control group and overall lower in the EHV 1&4 group compared to the EHV 1 group.

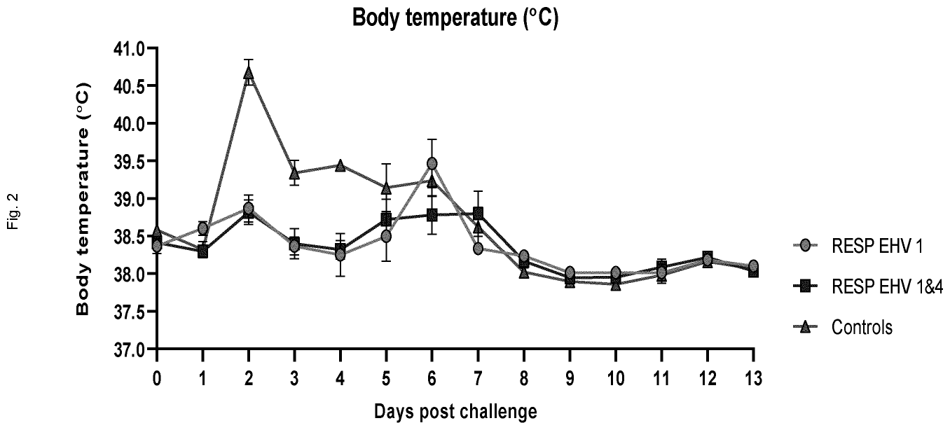

There was an increase in body temperature in the control group due to challenge from 2-7 d.p.c. peaking on 2 d.p.c. with an average temperature of 40.7° C. (FIG. 2). In the EHV 1 group a small increase in body temperature was observed on 2 d.p.c (on average 38.8° C.) and a peak on 6 d.p.c. (on average 39.5° C.). From 2-5 d.p.c. the increase in body temperature was clearly lower compared to the control group. In the EHV 1&4 group a small increase in body temperature was observed on 2 d.p.c (on average 38.8° C.) and 5-7 d.p.c. (on average 38.8° C.). From 2-6 d.p.c. the increase in body temperature was clearly lower compared to the control group. There was no notable difference in body temperature between both vaccinated groups, except on 6 d.p.c when the EHV 1 group had a higher temperature compared to the EHV 1&4 group. FIG. 2 outlines the body temperature.

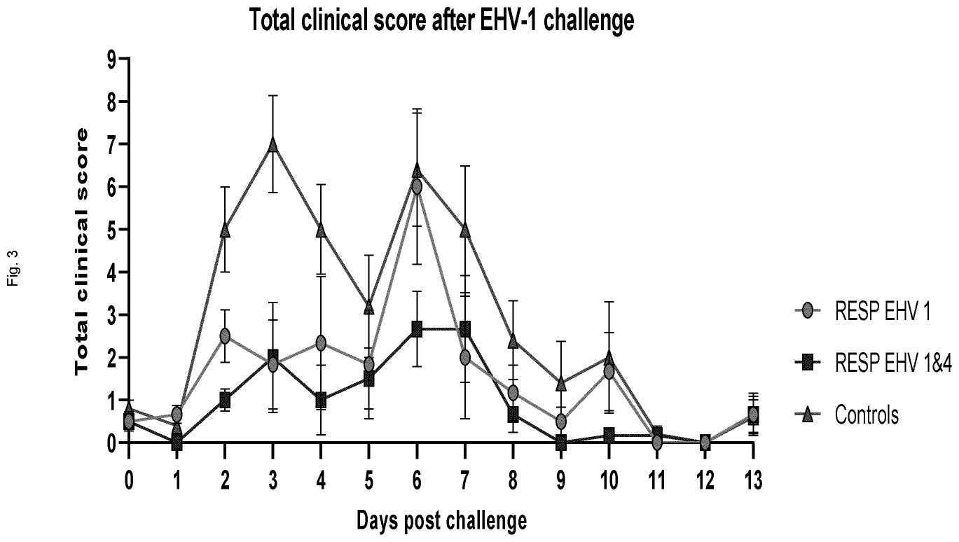

The sum of the body temperature score with the clinical score resulted in the total clinical score (FIG. 3). The total clinical score was clearly lower in both vaccinated groups compared to the control group, except for the EHV 1 group on 6 d.p.c. Between both vaccinated groups a clear difference in total clinical score was observed on 2, 4, 6 and 10 d.p.c. where the clinical score was higher in the EHV 1 group compared to the EHV 1&4 group.

Overall, the clinical score was 40% lower in the EHV 1 group and 70% lower in the EHV 1&4 group when compared to the control group. The overall clinical score of the EHV 1&4 group was 50% lower when compared to the EHV 1 group. Also, it can be seen that the peaks of clinical score was lower in the EHV 1&4 group when compared to the EHV 1 group. The severity of the clinical reactions as expressed by the height of the peak, in the control group and the EHV 1 group was comparable (4.8 vs 4.3) whereas the peak for the EHV 1&4 was only 1.7 which is a reduction of 65% when compared to control. The peak of the clinical score of the EHV 1&4 group was 60% lower than the peak of the clinical score of the EHV 1 group. Clinical score is shown in FIG. 1

In FIG. 1 it can be seen that whereas the control group and the EHV 1 vaccinated group had 3 peaks in the clinical score at day 3, 6 and 10. However the EHV 1&4 vaccinated group only had two peaks at day 3 and 6. After day 8 no clinical signs were recorded for the EHV 1&4 group.

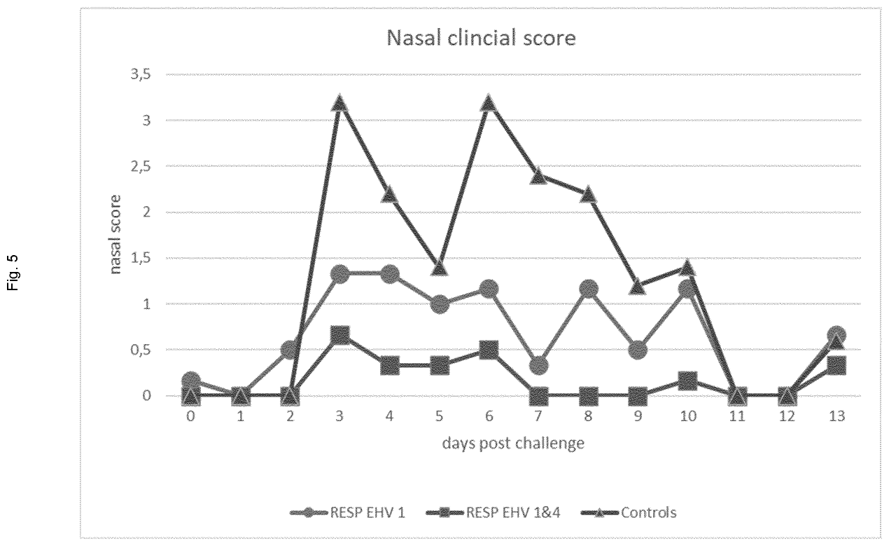

In particular the nasal score was lower in the EHV 1&4 group when compared to the EHV 1 and to the control group, see FIG. 5 Whereas the EHV 1 vaccinated animals showed a decrease of nasal clinical signs of 48%, the decrease of EHV 1&4 vaccinated animals was 87% when compared to non-vaccinated animals. When compared to EHV 1 vaccinated animals, the EHV 1&4 vaccinated animals had 75% less nasal clinical reaction. Also in general the severity of the nasal reactions was a lot lower for the vaccinated animals when compared to the non-vaccinated animals. The EHV 1 vaccinated animals had 60% lower clinical reaction than non-vaccinated animals, and the EHV 1&4 vaccinated animals had even 80% less severe clinical reactions than non-vaccinated animals. When compared to EHV 1 vaccinated animals, the EHV 1&4 vaccinated animals had 50% less severe nasal clinical reactions.

### Viremia

Heparin blood samples were kept on ice in the laboratory and used for PBL isolation according to standard procedure. Isolated PBL were analysed for the presence of EHV.

Presence of EHV was assessed by analysing isolated PBL before each vaccination and before and after EHV-1 challenge.

In short, PBL are isolated using a density gradient such as ACCUSPIN System-Histopaque-1077. After counting, the PBL are inoculated on VERO cells (e.g. Marburg) and/or RK-13 cells. VERO cells are used to detect EHV-4 and EHV-1 virus. RK-13 cells are used to detect EHV-1 virus. The monolayer will be examined microscopically for specific CPE caused by EHV-1 (+ and − scoring) after 8-10 days.

All horses were negative for EHV-1 and EHV-4 up to time of challenge. After challenge there was a difference in the percentage of animals positive for EHV-1 between the three groups. In both vaccinated groups the percentage of positive horses was lower compared to the controls and in the EHV 1&4 group the percentage of positive animals was lower compared to the EHV 1 group.

Viremia data was analysed as follows, post-challenge positive Peripheral blood lymphocytes (PBL) isolation data (1 for positive and 0 for negative) tested using 10⁵, 10⁶, and 10⁷ cell culture will be given 3, 2, and 1 weights (3 for positive PBL tested in 10⁵ culture, 2 for positive PBL tested in 10⁶ culture, 1 for positive PBL tested in 10⁷ culture, 0 for negative PBL in all situations). This weighted score was used to take the different PBL dilutions and the impact of these dilution on the chance of finding positive PBL into account.

Subsequently, the summation of the three values (the PBL weighted score) per animal per time period were analyzed by means of GEE using a cumulative logit model. In addition, the Area under the curve (AUC) of the PBL weighted score were calculated by the linear trapezoidal rule and analyzed by ANOVA, providing an indication of the amount of virema over the period post challenge.

AUC was 9 for EHV-1, 5 for EHV 1&4, and 18.4 for the control group. It is clear that EHV 1&4 reduced EHV-1 viremia the most by almost half when compared to the EHV-1 vaccinated group and by almost 4 when compared to the control group. The viremia for EHV 1&4 was 75% reduced when compared to controls and 45% reduced when compared to EHV-1 vaccinated animals. Viremia of EHV-1 vaccinated animals was 50% lower when compared to controls. It should be noted that the dose of EHV-1 in the EHV 1&4 group was 8 times lower than the dose of EHV-1 in the EHV 1 group.

When looking at peak viremia, the height of the peak for the controls was 4.8, for EHV 1 it was 2 and for EHV 1&4 1.2. So the height of the peak of the viremia for animals vaccinated with EHV 1&4 was reduced by 40% when compared to EHV 1 vaccinated animals and reduced by 75% when compared to control animals.

In addition, the number of animals positive for EHV-1 viremia was lower in animals vaccinated with EHV 1&4 when compared to controls and animals vaccinated with EHV 1 but not EHV 4. The below table 4 shows the percentage of animals that was positive in the cell culture with 10⁶ PBL.

TABLE 4
Percentage of horses positive for EHV-1

| Days post challenge | 0 | 2 | 4 | 6 | 8 | 10 | 13 |
|---|---|---|---|---|---|---|---|
| RESP EHV 1 | 0 | 0 | 33 | 50 | 17 | 0 | 0 |
| RESP EHV 1&4 | 0 | 0 | 33 | 17 | 0 | 0 | 0 |
| Control | 0 | 0 | 100 | 100 | 20 | 0 | 0 |

The table shows that the percentage of animals positive for viremia is 33% for EHV 1&4 vaccinated animals, 50% for animals vaccinated with EHV 1 but not EHV 4 and 100% for non-vaccinated animals. Also it can be seen that the virus is much faster cleared from the blood than for the controls and EHV-1 vaccinated group.

EHV 1&4 protected horses against an EHV-1 challenge as good and often even better than the EHV 1 vaccination whereas the dose of EHV-1 in the EHV 1&4 vaccine was significantly lower than in the EHV 1 vaccine. Also EHV 1&4 provided a reduced viremia when compared to control group and when compared to a EHV 1 vaccinated group.

1. An immunogenic composition comprising an antigen of Equine Herpes Virus type 1 (EHV-1) and an antigen of Equine Herpes Virus type 4 (EHV-4), wherein the ratio of EHV-1:EHV-4 antigen is at least 1:1.1.

2. The immunogenic composition according to claim 1 wherein the antigen is an inactivated EHV-1 and inactivated EHV-4.

3. The immunogenic composition according to claim 1 wherein the immunogenic composition comprises a dose of EHV-1 antigen between 500 and 10.000 EU of EHV 1.

4. The immunogenic composition according to claim 1 wherein the immunogenic composition comprises a dose of EHV-4 antigen between 2500 and 40.000 EU of EHV 4.

5. The immunogenic composition according to claim 1 wherein the composition further comprises an adjuvant.

6. A method of reducing viremia of EHV-1 in an animal comprising administering to the animal the immunogenic composition of claim 1.

7. (canceled)

8. The method of claim 6, wherein the animal is an equine.

9. The method of claim 6, wherein the animal is administered with at least two doses of the immunogenic composition.

10. The method of claim 9, wherein the animal is administered with at least two doses of the immunogenic composition and wherein the doses are administered at least 15 days apart.

11. The method of claim 9, wherein the animal is administered with at least two doses of the immunogenic composition, wherein the two doses are followed by administration of a further dose of the immunogenic composition wherein the further dose is administered 3 to 24 month after the second dose.

12. The method of claim 8, wherein the animal is a pregnant mare.

13. The method of claim 12, wherein the animal is administered at least three doses of the immunogenic composition.

14. The method of claim 12, wherein the animal is administered the immunogenic composition during month 5, 7, and/or 9 of gestation.

15. The method of claim 6, wherein the administration of the immunogenic composition is intramuscular or intranasal.

16. The immunogenic composition of claim 2, wherein the inactivated EHV-1 and inactivated EHV-4 are inactivated by an inactivating agent selected from the group consisting of formaldehyde and bromoethylamine hydrobromide.

17. The immunogenic composition of claim 5, wherein the adjuvant is a saponin.

18. The method of claim 8, wherein the animal is a horse.

19. The method of claim 18, wherein the horse is a foal of at least 3 month of age or a pregnant mare.
